(19) United States
(12) Patent Application Publication
(10) Pub. No.: US 2025/0185982 A1
(43) Pub. Date: Jun. 12, 2025

(54) SKIN SENSITIVITY OR RASH DETECTOR

(71) Applicant: Senseonics, Incorporated, Germantown, MD (US)

(72) Inventor: Ravi Rastogi, Columbia, MD (US)

(73) Assignee: Senseonics, Incorporated, Germantown, MD (US)

(21) Appl. No.: 19/046,023

(22) Filed: Feb. 5, 2025

Related U.S. Application Data
(62) Division of application No. 17/097,566, filed on Nov. 13, 2020.
(60) Provisional application No. 62/934,593, filed on Nov. 13, 2019.

(51) Int. Cl. A61B 5/00; A61B 5/145; A61B 5/1455

(52) U.S. Cl. CPC A61B 5/445; A61B 5/0077; A61B 5/1451; A61B 5/14532; A61B 5/1455; A61B 5/6833

(57) ABSTRACT

Systems, methods, and devices for determining the condition of a skin surface covered by an adhesive patch. An external device may be configured to be attached to the skin surface via the adhesive patch. A light source of the external device may emit light through the adhesive patch. A photodetector of the device may output a signal indicative of an amount of the light received by the photodetector after passing through the adhesive patch. A controller of the device may determine, based on the signal, a condition of the skin surface covered by the adhesive patch. In some alternative embodiments, the photodetector may instead by in an implantable device, which may convey the signal to the external device. In some other alternative embodiments, the light source may instead be in an implantable device.

### CROSS-REFERENCE TO RELATED APPLICATION

The present application is a divisional of U.S. application Ser. No. 17/097,566, filed on Nov. 13, 2020, which claims the benefit of priority to U.S. Provisional Application Ser. No. 62/934,593, filed on Nov. 13, 2019, which are incorporated herein by reference in their entireties.

### BACKGROUND

### Field of Invention

The present invention relates generally to detecting skin rash and/or skin sensitivity underneath an adhesive patch. Specifically, aspects of the present invention may relate to an external device that is held in place on the skin surface by the adhesive patch and detects skin rash and/or skin sensitivity underneath an adhesive patch. More specifically, aspects of the present invention may relate to detecting skin rash and/or skin sensitivity using an analyte monitoring system including an implantable device and the external device.

### Discussion of the Background

External devices may be used for a variety of purposes. For example, in an analyte monitoring system, an external device may power an implantable device and/or receive measurements (e.g., light and/or temperature measurements) from the implantable device. In some instances, the external device may be attached to a skin surface of a living animal via an adhesive patch. In some instances, the adhesive patch may cause skin irritation and/or redness. The skin irritation is often detected via a visual inspection after the adhesive patch is removed. However, detection of skin irritation after the adhesive patch is removed is often too late for preventative measures.

There is presently a need in the art for improved detection of skin rash and/or skin sensitivity underneath an adhesive patch.

### SUMMARY

A potential adverse effect of attaching an external device to a skin surface is skin irritation. In other words, attaching an external device to a skin surface (e.g., for analyte monitoring) may cause skin irritation on the skin surface or have an adverse effect on skin sensitivity. Early detection of the skin irritation and/or skin rash (e.g., detection while the adhesive patch is still on) may allow preventative measures (e.g., the application of a topical cream).

One aspect of the invention may provide an external device configured to be attached to a skin surface via an adhesive patch. The device may include a light source, a photodetector, and a controller. The light source may be configured to emit light through the adhesive patch. The photodetector may be configured to output a signal indicative of an amount of the light received by the photodetector after passing through the adhesive patch. The controller may be configured to determine, based on the signal, a condition of the skin surface covered by the adhesive patch.

In some aspects, the light source may be a first light source, the light may be first light, the photodetector may be a first photodetector, the signal may be a first signal, the device may further include a second light source configured to emit second light through the adhesive patch, the device may further include a second photodetector configured to output a second signal indicative of an amount of the second light received by the second photodetector after passing through the adhesive patch, and the controller may be configured to determine, based on the first and second signals, the condition of the skin surface covered by the adhesive patch.

Another aspect of the invention may provide a system including an external device, an implantable device, and an adhesive patch. The external device may include a light source and a controller. The implantable device may include a photodetector. The adhesive patch may be configured to attach the external device to a skin surface. The light source may be configured to emit light through the adhesive patch. The photodetector may be configured to output a signal indicative of an amount of the light received by the photodetector after passing through the adhesive patch and the skin surface. The implantable device may be configured to convey the signal. The external device may be configured to receive the conveyed signal. The controller of the external device may be configured to determine, based on the received signal, a condition of the skin surface covered by the adhesive patch.

In some aspects, the light source may be a first light source, the light may be first light, the photodetector may be a first photodetector, the signal may be a first signal, the external device may further include a second light source configured to emit second light through the adhesive patch, the implantable device may further include a second photodetector configured to output a second signal indicative of an amount of the second light received by the second photodetector after passing through the adhesive patch, and the controller may be configured to determine, based on the first and second signals, the condition of the skin surface covered by the adhesive patch.

Yet another aspect of the invention may provide a system including an external device, an implantable device, and an adhesive patch. The external device may include a photodetector and a controller. The implantable device include a light source. The adhesive patch may be configured to attach the external device to a skin surface. The light source may be configured to emit light. The photodetector may be configured to output a signal indicative of an amount of the light received by the photodetector after passing through the skin surface and the adhesive patch. The controller of the external device may be configured to determine a condition of the skin surface based on the signal.

In some aspects, the light source may be a first light source, the light may be first light, the photodetector may be a first photodetector, the signal may be a first signal, the implantable device may further include a second light source configured to emit second light, the external device may further include a second photodetector configured to output a second signal indicative of an amount of the second light received by the second photodetector after passing through the skin surface and the adhesive patch, and the controller may be configured to determine, based on the first and second signals, the condition of the skin surface covered by the adhesive patch.

Still another aspect of the invention may provide a method including using a light source of a device to emit light through an adhesive patch holding the device on a skin surface. The method may include using a photodetector of the device to output a signal indicative of an amount of the light received by the photodetector after passing through the adhesive patch. The method may include using a controller configured to determine, based on the signal, a condition of the skin surface covered by the adhesive patch.

In some aspects, the light source may be a first light source, the light may be first light, the photodetector may be a first photodetector, and the signal may be a first signal. The method may further include using a second light source of the device to emit second light through the adhesive patch. The method may further include using a second photodetector of the device to output a second signal indicative of an amount of the second light received by the second photodetector after passing through the adhesive patch. The method may further include using the controller configured to determine, based on the first and second signals, a condition of the skin surface covered by the adhesive patch.

Yet another aspect of the invention may provide a method including using a light source of an external device to emit light through an adhesive patch attaching the external device to a skin surface. The method may include using a photodetector of an implantable device to output a signal indicative of an amount of the light received by the photodetector after passing through the adhesive patch and the skin surface. The method may include using the implantable device to convey the signal. The method may include using the external device to receive the conveyed signal. The method may include using a controller of the external device to determine, based on the received signal, a condition of the skin surface covered by the adhesive patch.

In some aspects, the light source may be a first light source, the light may be first light, the photodetector may be a first photodetector, and the signal may be a first signal. The method may further include using a second light source of the external device to emit light through the adhesive patch. The method may further include using a second photodetector of the implantable device to output a second signal indicative of an amount of the second light received by the second photodetector after passing through the adhesive patch and the skin surface. The method may further include using the implantable device to convey the second signal. The method may further include using the external device to receive the conveyed second signal. The controller of the external device may determine the condition of the skin surface covered by the adhesive patch based on the received first and second signals.

Still another aspect of the invention may provide a method including using a light source of an implantable device to emit light. The method may include using a photodetector of an external device to output a signal indicative of an amount of the light received by the photodetector after passing through a skin surface and an adhesive patch. The method may include using a controller of the external device to determine a condition of the skin surface based on the signal.

In some aspects, the light source may be a first light source, the light may be first light, the photodetector may be a first photodetector, and the signal may be a first signal. The method may further include using a second light source of the implantable device to emit second light. The method may further include using a second photodetector of the external device to output a second signal indicative of an amount of the second light received by the second photodetector after passing through the skin surface and the adhesive patch. The controller may determine the condition of the skin surface based on the first and second signals.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1A is a schematic view illustrating a system according to some embodiments.

FIG. 1B is a schematic view illustrating an implantable device and an external device of a system according to some embodiments.

FIG. 2 is a back view of an external device including first and second light sources and first and second light detectors according to some embodiments.

FIG. 3 is a schematic view illustrating an implantable device and an external device of a system according to some embodiments.

FIG. 4 is a flow chart illustrating a skin surface condition determining process according to some embodiments.

FIG. 5 is a flow chart illustrating a skin surface condition determining process according to some embodiments.

FIG. 6 is a flow chart illustrating a skin surface condition determining process according to some embodiments.

### DETAILED DESCRIPTION OF PREFERRED EMBODIMENTS

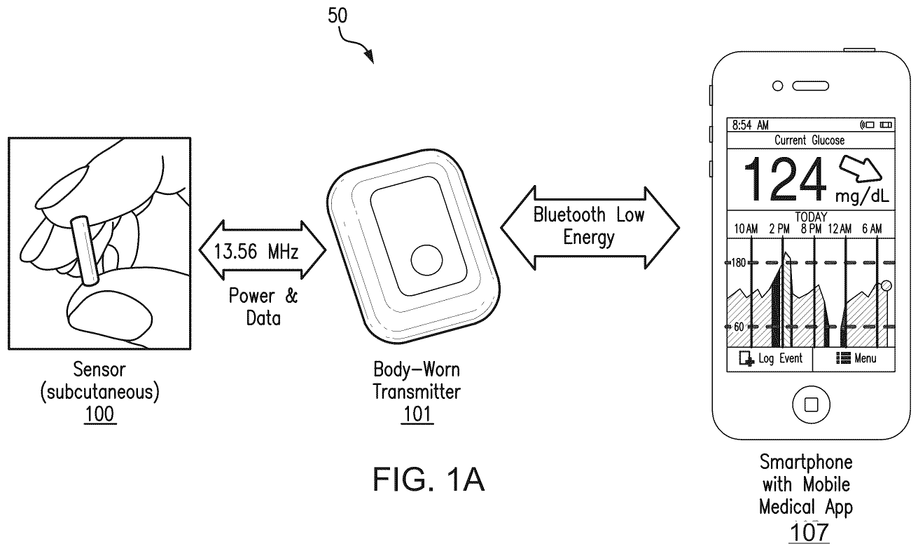

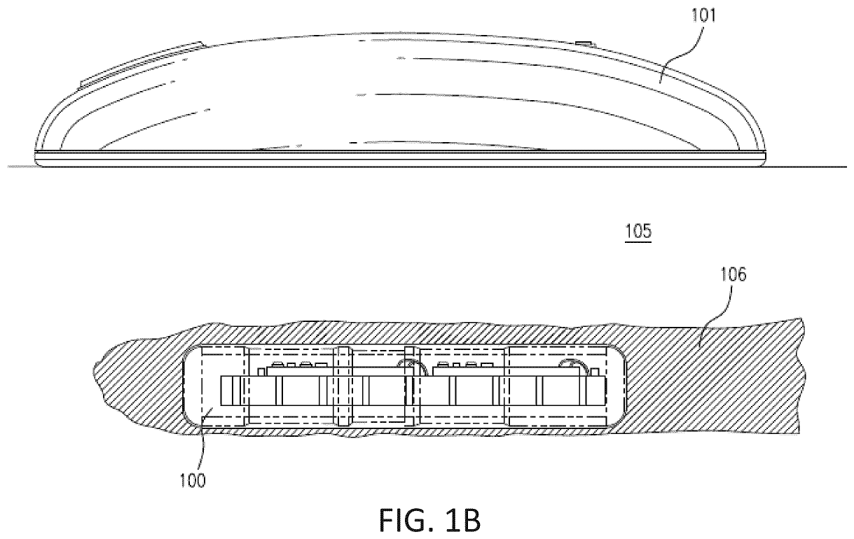

FIG. 1A is a schematic view of an exemplary system 50 according to some embodiments. In some non-limiting embodiments, the system 50 may be an analyte monitoring system. In some non-limiting embodiments, the system 50 may be a continuous analyte monitoring system (e.g., a continuous glucose monitoring system). In some embodiments, the system 50 may include one or more of an implantable device 100, an external device 101, and a display device 107. In some embodiments, the implantable device 100 may be an analyte sensor. In some non-limiting embodiments, the implantable device 100 may be a small, fully subcutaneously implantable sensor that measures the amount or concentration of an analyte (e.g., glucose) in a medium (e.g., interstitial fluid) of a living animal (e.g., a living human). However, this is not required, and, in some alternative embodiments, the implantable device 100 may be a partially implantable (e.g., transcutaneous) device. In addition, although embodiments of the invention are described with respect to an analyte monitoring system in which the implantable device 100 is an analyte sensor, this is not required. In some alternative embodiments, the implantable device 100 is not a sensor and is instead a different type of implantable device, such as, for example and without limitation, an insulin pump, pacemaker, or electrical/heat therapy device.

In some embodiments, the external device 101 may be an externally worn device (e.g., attached via an armband, wristband, waistband, or adhesive patch). In some embodiments, the external device 101 may remotely power and/or communicate with the implantable device 100 (e.g., via near field communication (NFC)). However, this is not required, and, in some alternative embodiments, the external device 101 may power and/or communicate with the implantable device 100 via one or more wired connections. In some embodiments, the external device 101 may power and/or communicate with the implantable device 100 to initiate and receive the measurements from the implantable device 100. In some embodiments, the external device 101 may be a transceiver. In some non-limiting embodiments, the external device 101 may be a smartphone (e.g., an NFC-enabled smartphone). In some embodiments, the external device 101 may communicate information (e.g., one or more analyte measurements) wirelessly (e.g., via a Bluetooth™ communication standard such as, for example and without limitation Bluetooth Low Energy) to a hand held application running on a display device 107 (e.g., smartphone).

In some non-limiting embodiments, as illustrated in FIG. 1B, when the system 50 is in use, the implantable device 100 may be implanted in the tissue 105 of the living animal, and the external device 101 may be external to the tissue 105. In some embodiments, the back of the external device 101 may be adjacent to the tissue 105 (e.g., adjacent to the skin of the living animal). As shown in FIG. 1B, after implantation, the implantable device 100 may rest in a pocket 106 in the tissue 105, and the pocket 106 may surround the implantable device 100. In some non-limiting embodiments, the pocket 106 may be created by a tissue dissector tool before implantation of the implantable device 100 or by the implantation process.

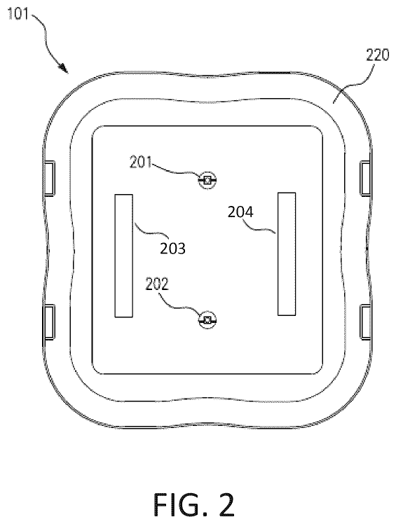

FIG. 2 is a back view illustrating an external device 101 embodying aspects of the present invention. In some embodiments, as shown in FIG. 2, the external device 101 may include a housing 220, which may be a back housing. In some embodiments, as shown in FIG. 2, the external device 101 may include one or more first light sources 201, one or more first light detectors 203, one or more second light sources 202, and/or one or more second light detectors 204.

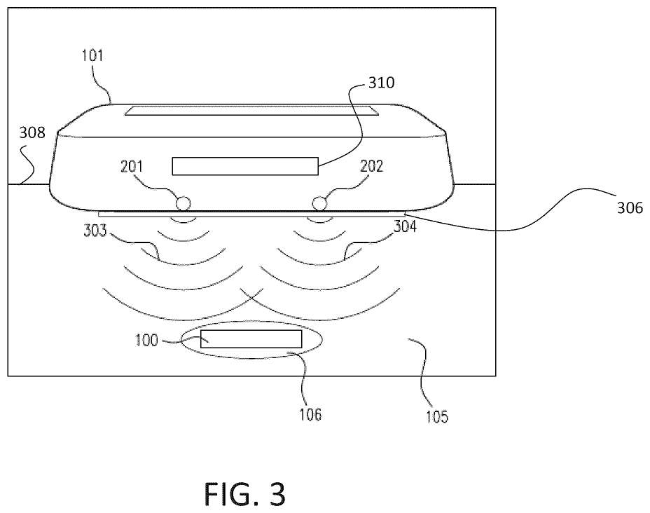

FIG. 3 is a schematic view illustrating an implantable device 100 and an external device 101 of a system 50 embodying aspects of the present invention. In some embodiments, as shown in FIG. 3, the external device of the system 50 may be attached to a skin surface 308 of a user via an adhesive patch 306.

In some embodiments, as shown in FIG. 3, the first light source 201 may emit a first light 303. In some embodiments, the first light 303 may be over a first wavelength range. In some embodiments, as shown in FIG. 3, the one or more second light sources 202 may emit a second light 304. In some embodiments, the second light 304 may be over a second wavelength range, which may be different than the first wavelength range. In some non-limiting embodiments, the first and second wavelength ranges do not overlap, but this is not required, and, in some alternative embodiments, the first and second wavelength ranges may overlap.

In some non-limiting embodiments, at least a portion of the first light 303 and/or the second light 304 may pass through the adhesive patch 306. In such embodiments, portions of the first light 303 and/or the second light 304 (hereinafter referred to as the “reflected first and/or second light 303, 304) may reflect off of the skin surface 308 and pass through the adhesive patch 306 towards the external device 101. The one or more first light detectors 203 and/or the one or more second light detectors 204 may be configured to receive the reflected first and/or second light 303, 304. In some embodiments, the one or more first light detectors 203 may be configured to detect the reflected first light 303 and the one or more second light detectors 204 are configured to detect the reflected second light 304. In some alternative embodiments, any combination of the one or more first and second light detectors 203, 204 may be used to detect the reflected first and/or second light 303, 304.

In some embodiments, IR light from the external device 101 may go right through the patch, and differences in the detected IR light may provide information about changes to the skin under the patch. In some embodiments, the first and second light detectors 203, 204 may be embedded in the external device 101 to detect the IR light, and a user can be alerted early if their skin is reacting to the patch.

In some non-limiting embodiments, the first light 303 may include one or more of red light and infrared light. In non-limiting embodiments, the first light 303 may include red light. In some non-limiting embodiments, the second light 304 may include one or more of red light and infrared light. In non-limiting embodiments, the second light 304 may include infrared light. In some non-limiting embodiments, the first and second wavelength ranges may be centered at, for example and without limitation, (a) 660 nm and 800 nm, respectively, (b) 750 nm and 850 nm, respectively, (c) 800 nm and 840 nm, respectively, (d) 654 and 740, respectively, (e) 740 nm and 800 nm, respectively, or (f) 660 nm and 940 nm, respectively.

In some alternative embodiments, the implantable device 100 may comprise the one or more first light sources 201 and/or the one or more second light sources 202. In such embodiments, one or more first light sources 201 and/or one or more second light sources 202 of the implantable device 100 may emit the first light 303 and/or the second light 304 towards the external device 101. In some embodiments, at least a portion of the first light 303 and/or the second light 304 may pass through the adhesive patch 306. In such embodiments, one or more first light detectors 203 and/or one or more second light detectors 204 on the external device 101 may be configured to receive at least the portion of the first light 303 and/or the second light 304 that passed through the adhesive patch 306. In some embodiments, the one or more first light detectors 203 may be configured to detect at least the portion of the first light 303, and the one or more second light detectors 204 may be configured to detect at least the portion of the second light 304. In some alternative embodiments, any combination of the one or more first and second light detectors 203, 204 may be used to detect at least the portion of the first and/or second light 303, 304.

In some alternative embodiments, the implantable device 100 may comprise the one or more first light detectors 203 and/or the one or more second light detectors 204. In such embodiments, one or more first light sources 201 and/or one or more second light sources 202 of the external device 101 may emit first light 303 and/or second light 304 towards the implantable device 100. In some embodiments, at least a portion of the first light 303 and/or the second light 304 may pass through the adhesive patch 306. In such embodiments, the one or more first light detectors 203 and/or the one or more second light detectors 204 on the implantable device 101 may be configured to receive at least the portion of the first light 303 and/or the second light 304 that passed through the adhesive patch 306. In some embodiments, the one or more first light detectors 203 may be configured to detect at least the portion of the first light 303, and the one or more second light detectors 204 may be configured to detect at least the portion of the second light 304. In some alternative embodiments, any combination of the one or more first and second light detectors 203, 204 may be used to detect at least the portion of the first and/or second light 303, 304. In some embodiments, the implantable device 100 may be configured to output a signal indicative of an amount of light received by the one or more first light detectors 203 and/or the one or more second light detectors 204,

In some embodiments, the external device 101 may include a peripheral interface controller (PIC) controller 310 and memory (e.g., Flash memory), which may be non-volatile and/or capable of being electronically erased and/or rewritten. The PIC controller 310 may control the overall operation of the external device 101. For example, the PIC controller 310 may control processing of data received via the one or more first light detectors 203 and/or the one or more second light detectors 204 or processing a signal conveyed by the implantable device 100 where the signal indicates an amount of light received by the one or more first light detectors 203 and/or the one or more second light detectors 204 on the implantable device 100.

In some embodiments, implantable device 100 may include a peripheral interface controller (PIC) controller and memory (e.g., Flash memory), which may be non-volatile and/or capable of being electronically erased and/or rewritten. The PIC controller may control the overall operation of the implantable device 100. For example, the PIC controller may control processing of data received via the one or more first light detectors 203 and/or the one or more second light detectors 204 and output a signal indicative of an amount of light received by the one or more first light detectors 203 and/or the one or more second light detectors 204.

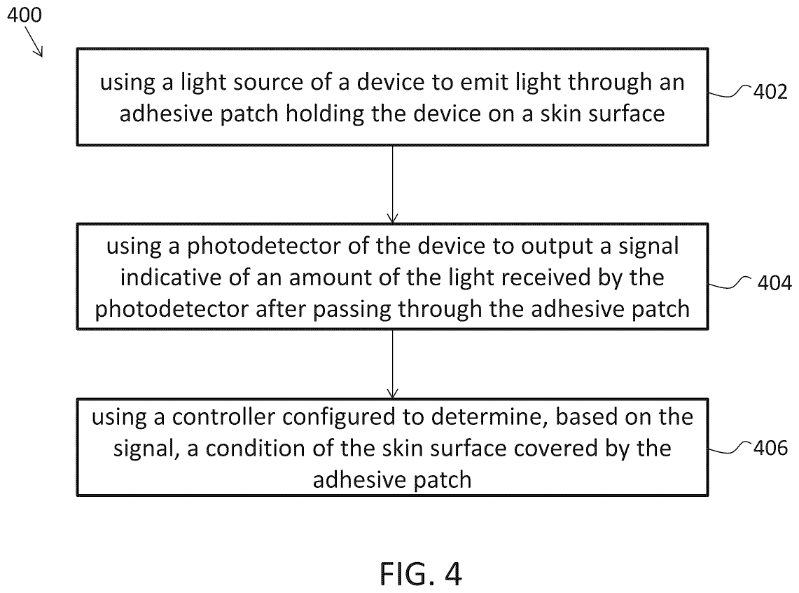

FIG. 4 is a flow chart illustrating a skin surface condition determining process 400 according to some embodiments. In some embodiments, one or more steps of the process 400 may be performed by an analyte monitoring system, such as, for example, the analyte monitoring system 50. In some embodiments, the process 400 may include a step 402 of using a light source (e.g., one or more first light sources 201) of the external device 101 to emit light (e.g., first light 303) through an adhesive patch 306 holding the device 101 on a skin surface 308. In some embodiments, the process 400 may include a step 404 of using a photodetector (e.g., one or more first light detectors 203) of the device 101 to output a signal indicative of an amount of the light received by the photodetector after passing through the adhesive patch 306. In some embodiments, the process 400 may include a step 406 of using a controller (e.g., the PIC controller 310) configured to determine, based on the signal, a condition of the skin surface 308 covered by the adhesive patch 306.

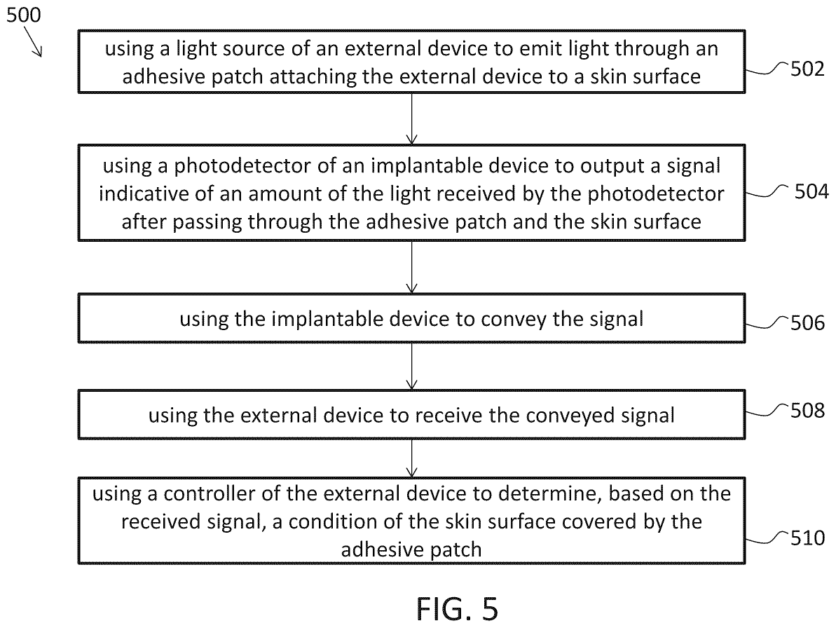

FIG. 5 is a flow chart illustrating a skin surface condition determining process 500 according to some embodiments. In some embodiments, one or more steps of the process 500 may be performed by an analyte monitoring system, such as, for example, the analyte monitoring system 50. In some embodiments, the process 500 may include a step 502 of using a light source (e.g., one or more first light sources 201) of an external device 101 to emit light (e.g., first light 303) through an adhesive patch 306 attaching the external device 101 to a skin surface 308. In some embodiments, the process 500 may include a step 504 of using a photodetector of an implantable device 100 to output a signal indicative of an amount of the light received by the photodetector after passing through the adhesive patch 306 and the skin surface 308. In some embodiments, the process 500 may include a step 506 of using the implantable device 100 to convey the signal. In some embodiments, the process 500 may include a step 508 of using the external device 101 to receive the conveyed signal. In some embodiments, the process 500 may include a step 510 of using a controller (e.g., the PIC controller 310) of the external device 101 to determine, based on the received signal, a condition of the skin surface 308 covered by the adhesive patch 306.

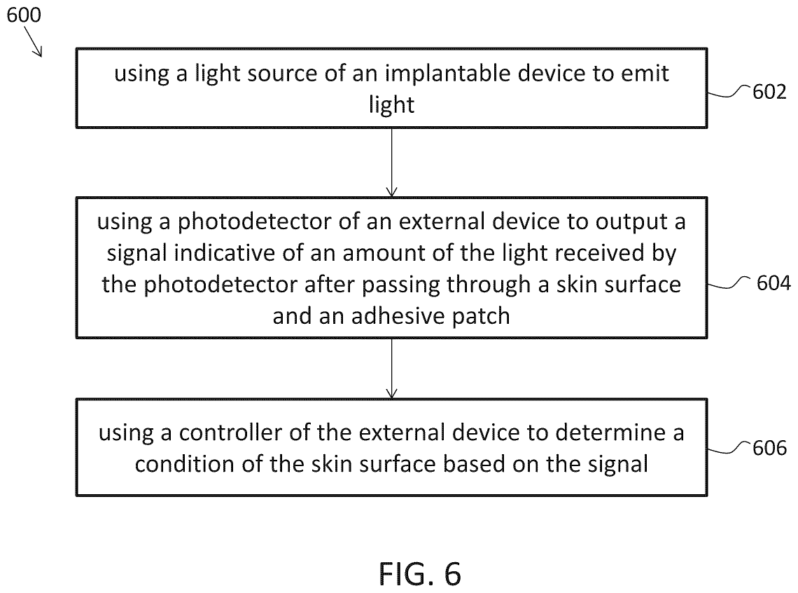

FIG. 6 is a flow chart illustrating a skin surface condition determining process 600 according to some embodiments. In some embodiments, one or more steps of the process 600 may be performed by an analyte monitoring system, such as, for example, the analyte monitoring system 50. In some embodiments, the process 600 may include a step 602 of using a light source of an implantable device 100 to emit light (e.g., first light 303). In some embodiments, the process 600 may include a step 604 of using a photodetector (e.g., one or more first light detectors 203) of an external device 101 to output a signal indicative of an amount of the light received by the photodetector after passing through a skin surface 308 and an adhesive patch 306. In some embodiments, the process 600 may include a step 606 of using a controller (e.g., the PIC controller 310) of the external device 101 to determine a condition of the skin surface 308 based on the signal.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. For example, although the invention has been described with respect to an external device in an analyte monitoring system, aspects of the present invention are applicable to any device attached to a skin surface using an adhesive patch. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A system comprising:
an external device including a controller;
an implantable device; and
an adhesive patch configured to attach the external device to a skin surface;
wherein:
one of the external device and the implantable device include a light source, and another of the external device and the implantable device include a photodetector;
the light source is configured to emit light through the adhesive patch and the skin surface;
the photodetector is configured to output a signal indicative of an amount of the light received by the photodetector after passing through the adhesive patch and the skin surface; and
the controller is configured to determine, based on at least the signal, a condition of the skin surface covered by the adhesive patch.

2. The system of embodiment 1, wherein:
the light source is a first light source;
the light is first light;
the photodetector is a first photodetector;
the signal is a first signal;
the one of the external device and the implantable device further includes a second light source configured to emit second light through the adhesive patch and the skin surface;
the other of the external device and the implantable device further includes a second photodetector configured to output a second signal indicative of an amount of the second light received by the second photodetector after passing through the adhesive patch and the skin surface; and
the controller is configured to determine, based on at least the first and second signals, the condition of the skin surface covered by the adhesive patch.

3. The system of claim 1, wherein the implantable device includes the photodetector and is configured to convey the signal, and the external device includes the light source and is configured to receive the conveyed signal.

4. The system of claim 1, wherein the implantable device includes the light source, and the external device includes the photodetector.

5. A method comprising:
using a light source of one of an external device and an implantable device to emit light through an adhesive patch, which attaches the external device to a skin surface, and the through the skin surface;
using a photodetector of another of the external device and the implantable device to output a signal indicative of an amount of the light received by the photodetector after passing through the adhesive patch and the skin surface; and
using a controller of the external device to determine, based on the signal, a condition of the skin surface covered by the adhesive patch.

6. The method of claim 5, wherein the light source is a first light source, the light is first light, the photodetector is a first photodetector, the signal is a first signal, and the method further comprises:
using a second light source of the one of the external device and the implantable device to emit light through the adhesive patch and the skin surface; and
using a second photodetector of the other of the external device and the implantable device to output a second signal indicative of an amount of the second light received by the second photodetector after passing through the adhesive patch and the skin surface;
wherein the controller of the external device determines the condition of the skin surface covered by the adhesive patch based on at least the first and second signals.

7. The method of claim 5, wherein the implantable device includes the photodetector, the external device includes the light source, and the method further comprises:
using the implantable device to convey the signal;
using the external device to receive the conveyed signal.

8. The method of claim 5, wherein the implantable device includes the light source, and the external device includes the photodetector.
